(19) United States
(12) Patent Application Publication
(10) Pub. No.: US 2016/0166136 A1
(43) Pub. Date: Jun. 16, 2016

(54) THERMAL MANAGEMENT FOR MEDICAL DEVICES AND RELATED METHODS OF USE

(71) Applicant: Boston Scientific Scimed, Inc., Maple Grove, MN (US)

(72) Inventors: Kirsten VIERING, Watertown, MA (US); Ryan FOSS, Gray, ME (US); Russell P. DRESHER, Hudson, MA (US)

(73) Assignee: Boston Scientific Scimed, Inc., Maple Grove, MN (US)

(21) Appl. No.: 14/950,043

(22) Filed: Nov. 24, 2015

Related U.S. Application Data
(60) Provisional application No. 62/089,970, filed on Dec. 10, 2014.

(51) Int. Cl. A61B 1/12; A61B 1/07; A61B 1/06

(52) U.S. Cl. CPC A61B 1/128; A61B 1/0684; A61B 1/07

(57) ABSTRACT

A medical device configured for insertion into a body may include an elongate member extending from a proximal end to a distal end, and the distal end may be configured to be positioned inside the body. The medical device also may include an optical fiber disposed in a lumen of the elongate member and also may include an illumination source configured to emit light through the optical fiber. The illumination source may be disposed on a first surface of a heat dissipating member. An oscillating member also may be included in the medical device. The oscillating member may face a second surface of the heat dissipating member and may be configured to direct air at the second surface of the heat dissipating member.

### CROSS-REFERENCE TO RELATED APPLICATIONS

This application claims the benefit of U.S. Provisional Application No. 62/089,970, filed Dec. 10, 2014, the disclosure of which is incorporated herein by reference in its entirety.

### TECHNICAL FIELD

Various aspects of the present disclosure relate generally to medical systems and devices. In particular, exemplary embodiments relate to endoscopic medical devices for visualization. Embodiments also cover methods of using such systems and devices.

### BACKGROUND

Medical devices are often inserted into the body to perform a therapeutic and/or diagnostic procedure inside a patient's body. An example of such a device is an endoscope, which is a flexible instrument introduced into the body for diagnostic or therapeutic purposes. Typically, endoscopic devices are inserted into the body through an opening (a natural opening or an incision), and are delivered to a work site inside the body through a body channel, such as, for example, the esophagus. Imaging devices incorporated in endoscopes allow endoscopists to view the work site from outside the body and remotely operate the endoscope to perform a desired diagnostic/therapeutic procedure at the work site. There are many different types of endoscopes in use today and embodiments of the current disclosure may be applied with any of these different types of endoscopes or other medical devices. In general, embodiments of the current disclosure may be applicable with any type of medical device that can be inserted into a body, and that allows an endoscopist outside the body to visualize a region inside the body. For the sake of brevity, however, the novel aspects of the current disclosure will be described with reference to an endoscope.

In a typical application, a distal end of an endoscope may be inserted into the body through an opening in the body. This opening may be a natural anatomic opening, such as, for example, the mouth, rectum, vagina, etc., or an incision made on the body. The endoscope may be pushed into the body such that the distal end of the endoscope proceeds from the point of insertion to a region of interest (work site) within the body by traversing a body channel. The endoscope may include one or more lumens extending longitudinally from the proximal end to the distal end of the endoscope. These lumens may deliver various diagnostic/treatment devices from outside the body to the work site to assist in the performance of the intended procedure at the work site.

Among others, these lumens may include an illumination lumen that may include an illumination source to illuminate a field of view at the work site, and/or an imaging lumen through which an imaging device may be inserted to capture an image of the work site and deliver the image outside the body for viewing.

Many sources of illumination may be used in endoscopes. Recently, light emitting diodes (LEDs) have been used due, in part, to their improved efficiency in converting electrical energy into photons over standard light sources used in endoscopy, like xenon or halogen. However, LEDs have a propensity to cause a temperature increase in areas in close proximity via conductive heating, especially at higher drive currents required to achieve greater light outputs.

In addition to conductive heating, the emitted light from LEDs also may cause radiative heating of other components in the endoscope, which may be near the LEDs. In particular, heat generated by the LEDs may cause radiative heating of illumination fibers and any connector that may house the illumination fibers of the endoscopes, as these may be placed in front of the LED, such that light can be coupled from the LED into the illumination fiber.

Maintaining sufficiently low temperatures at the LED is therefore important, for example, for the following reasons: the light output of the LED reduces as a function of temperature; high temperatures can affect the fibers placed in front of the connectors, leading to a reduced light coupling efficiency or even permanent damage (for example melting of plastic fibers); higher temperatures in the LED may limit the lifetime (in addition to the performance) of the LED; and an increase in the connector's temperature may potentially pose a safety hazard upon removal of the LED from the endoscope.

Therefore, effective thermal management may be implemented to maintain a compact design of the light source while sufficiently cooling the area surrounding the LED.

### SUMMARY

Examples of the present disclosure are related to, among other things, medical device for illuminating and visualizing internal areas of a subject's body. Each of the examples disclosed herein may include one or more of the features described in connection with any of the other disclosed examples.

In one example, a medical device may be configured for insertion into a body, and may comprise an elongate member extending from a proximal end to a distal end, the distal end may be configured to be positioned inside the body. The medical device also may include an optical fiber disposed in a lumen of the elongate member, and an illumination source configured to emit light through the optical fiber. The illumination source may be disposed on a first surface of a heat dissipating member. The medical device also may include an oscillating member, which may face a second surface of the heat dissipating member and may be configured to direct air at the second surface of the heat dissipating member.

Examples of the medical device may include one or more of the following features: The medical device may include at least one inlet which may be configured to receive air into the medical device and at least one outlet which may be configured to exhaust air from the medical device. The illumination source may comprise at least one LED. The heat dissipating member may include one or more protruding members extending between the first surface and the second surface and which may be configured to increase a surface area of the heat dissipating member. The medical device also may include a control member which may be configured to control characteristics of the oscillating member. The control member may be configured to receive data from a temperature sensor. A surface of the illumination source may be separated from a surface of the optical fiber by a space. The medical device also may include a second oscillating member which may be configured to direct air in the space separating the surface of the illumination source and the surface of the optical fiber. The second oscillating member may include a duct member which may be configured to focus the air in the space separating the surface of the illumination source and the surface of the optical fiber. The medical device also may include a connector disposed between the optical fiber and the illumination source and which may be coupled to a proximal end of the optical fiber. The medical device may be an endoscope.

In another example, a medical device may be configured for insertion into a body. The medical device may include an elongate member extending from a proximal end to a distal end, the distal end may be configured to be positioned inside the body. The medical device may include an optical fiber disposed in a lumen of the elongate member, and an illumination source configured to emit light through the optical fiber. The illumination source may be disposed on a first surface of a heat dissipating member. The medical device also may include a movable member configured to generate and direct air in a space between the illumination source and the optical fiber.

Examples of the medical device may include one or more of the following features. The heat dissipating member may include one or more protruding members extending between the first surface and a second surface and configured to increase a surface area of the heat dissipating member. The medical device also may include a duct coupled to the movable member and which may be configured to focus the air generated by the movable member. The medical device also may include least one inlet configured to receive air into the medical device and at least one outlet configured to exhaust air from the medical device. The medical device may include a control member configured to control characteristics of the oscillating member. The control member may be configured to receive data from a temperature sensor. The medical device also may include a second oscillating member disposed at an end portion of the heat-dissipating member and configured to direct air at the heat-dissipating member. In addition or alternatively, the medical device may include a connector disposed between the optical fiber and the illumination source and coupled to a proximal end of the optical fiber.

In another example, a method of dissipating heat from an illuminating medical device may comprise receiving air from outside the medical device through an inlet. The method may include generating a first airflow path from a first oscillating device to a first surface of an illumination source. The method also may include generating a second airflow path from a second oscillating device to a space between a second surface of the illumination source and an optical fiber, and expelling air from the medical device. The method may include one or more of the following features. The second surface of the illumination source may be coupled to a heat dissipating member. The heat dissipating member may include one or more protruding member extending from a surface of the heat dissipating member. At least one of the oscillating device may include a duct, and the duct may focus the air generated by the at least one of the oscillating devices. The method also may include adjusting characteristics of at least one of the oscillating devices. The adjusting of the characteristics of the oscillating devices may be done automatically based on temperature data.

It may be understood that both the foregoing general description and the following detailed description are exemplary and explanatory only and are not restrictive of the claimed features.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a schematic view of an embodiment of a medical device performing an exemplary medical procedure;

FIG. 2 is an illustration of the proximal portion of the medical device of FIG. 1;

FIG. 3 is an illustration of the distal end of the medical device of FIG. 1;

FIG. 4 is an illustration of an illumination portion of a medical device; and

FIG. 5 is an illustration of an illumination portion of a medical device according to another embodiment.

### DETAILED DESCRIPTION

### Overview

The present disclosure is drawn to medical devices and methods of using medical devices. Portions of the medical device may be used to provide illumination during a medical procedure. The medical device also may include components to dissipate heat generated during use of the illuminating portions of the medical device. For example, heat may be dissipated by forcing ambient air into a space between an illumination source and an optical fiber. Reference will now be made in detail to aspects of the present disclosure, examples of which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject.

Exemplary Aspects

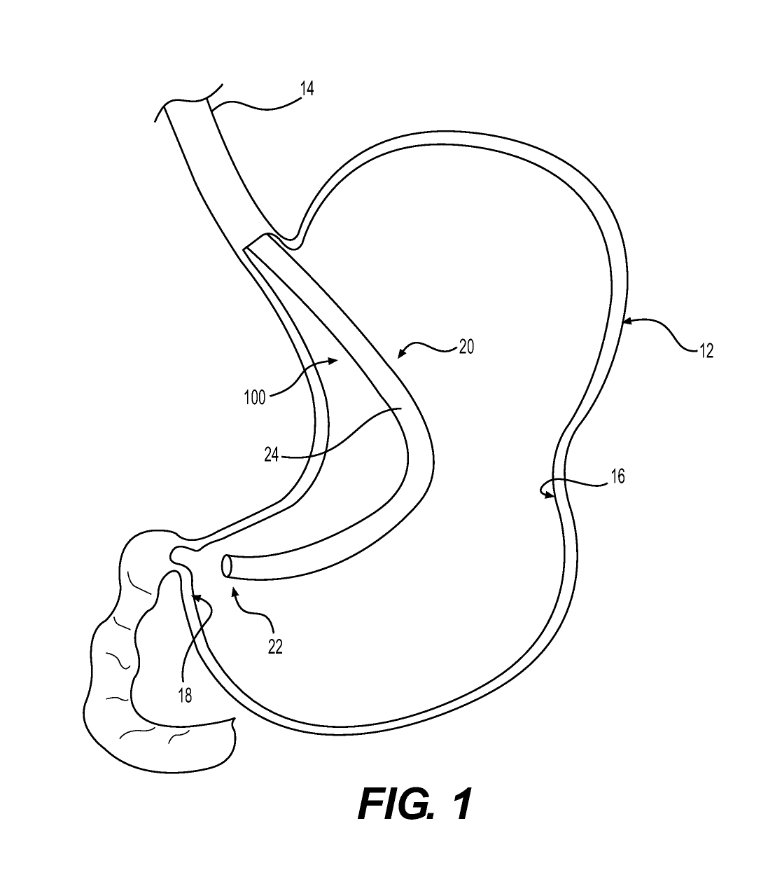

FIG. 1 illustrates a distal end 22 of an elongate member 20 of a medical device 100 during a medical procedure in the stomach 12. The distal end 22 of the elongated member 20 is shown positioned proximate a worksite 18 by the stomach wall 16. The distal end 22 of the elongate member 20 may be any part of any device suitable for insertion in the body such as an endoscope. The distal end 22 of the elongate member 20 may be introduced into the body via any suitable natural orifice (e.g. through the mouth and down the esophagus 14) or through a surgically created opening. One or more portions 24 of the elongate member 20, such as the distal end 22 may be flexible, and the shape and orientation of these portions may be controlled in any suitable manner, such as via control wires, hinges, and/or shape memory materials. The elongate member 20 also may be movable within the body in any direction in any suitable manner.

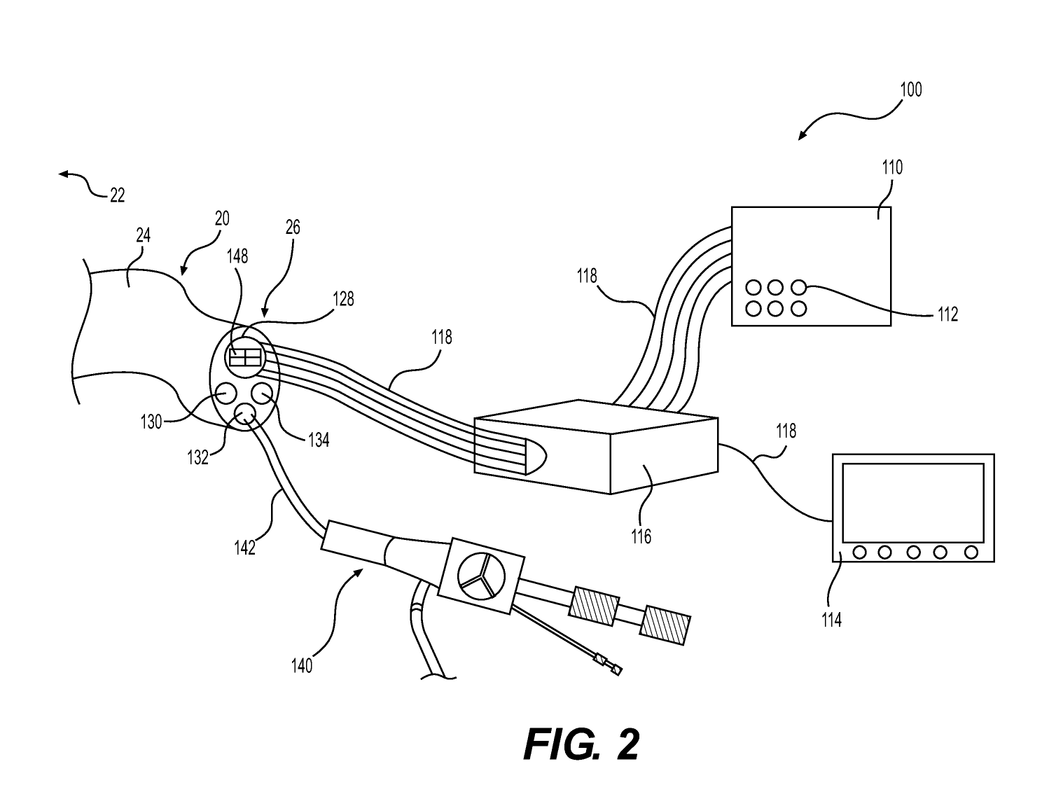

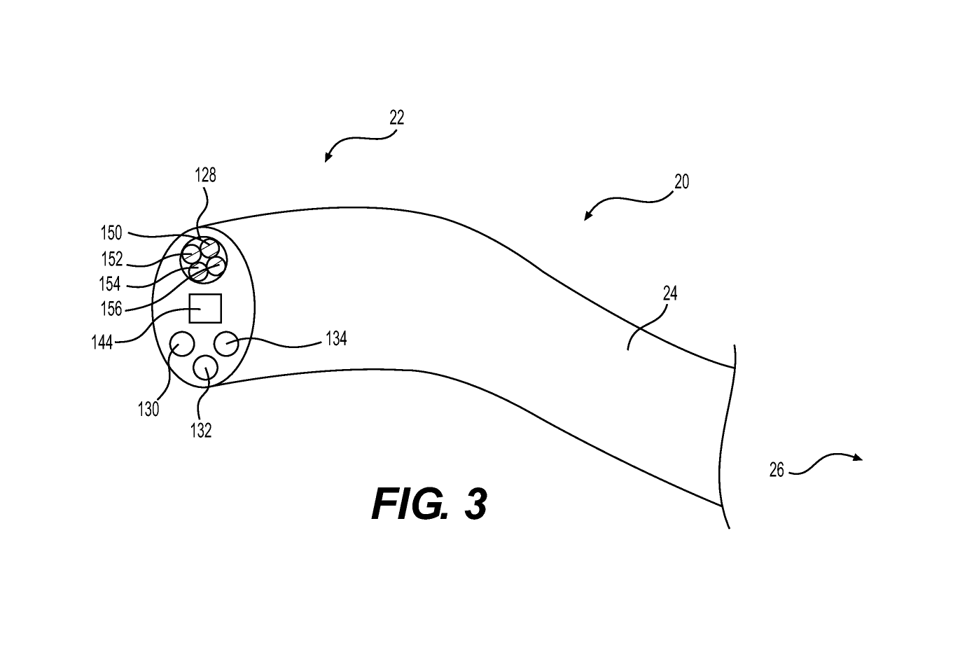

FIG. 2 illustrates a proximal end 26 of the elongate member 20 of the medical device 100. The elongate member 20 may include the flexible portion 24 and lumens 128, 130, 132, and 134 through which tools and instruments may be passed for accessing a worksite in the body. For example, a tool 140 may include a distal portion 142 that may be inserted through lumen 132 of the elongate member 20 to a worksite (e.g. worksite 18) to perform a therapeutic or diagnostic procedure. As shown in FIG. 2, the proximal end of the lumen 128 may include one or more illumination sources 148, which may provide illuminating light to the distal end 22 of the elongate member 20 via one or more optical fibers, such as optical fiber 150, 152, 154, and 156 (shown in FIG. 3 and discussed below). In some examples, the illumination source(s) 148 may be positioned proximate the proximal end 26 of the elongate member 20 (e.g. outside the elongate member 20), or in a handle portion of the medical device 100. The illumination source(s) 148 may receive power from a controller 116 via either wires 118 or any other suitable manner. In turn, the controller 116 may be coupled, either wirelessly or via wires 118 to an illumination controller 110 having one or more control buttons 112 for adjusting illumination characteristics of the illumination source(s) 148. It may be understood that although the controllers 110 and 116 are shown as separate components, they may be arranged in a single housing and/or the functions of both of the controllers 110 and 116 may be performed by either one of the controllers 110, 116.

The controllers 110 and 116 may be housed in a handle portion (not shown) of the medical device 100 and may include one or more processors, memory components, and other electronic circuitry to electronically control the illumination source 148.

As noted above, in some examples, as shown in FIG. 3, the device 100 may include multiple illumination sources 148, each of which may be connected to an optical fiber, such as optical fibers 150, 152, 154, and 156, which may be configured to transmit light generated from the illumination sources 148 out of the distal end 22 of the elongate member 20 to illuminate a location. In some examples, each of the optical fibers 150-156 may transmit light from separate LEDs and/or other illumination sources 148. The optical fiber 150-156 may have any suitable surface features, for example, the surfaces may be polished at the distal and/or proximal ends to maximize coupling efficiency and minimize Fresnel reflections. Illumination source 148 may have any suitable size and shape, including a flat top surface with glass over the surface. Each illumination source 148 providing light to the optical fibers 150-156 may be separately controlled, for example, by controllers 110 and/or 116, and configured to independently illuminate a location. In this manner, the illumination provided by each optical fiber 150-156 at the distal end 22 of the elongate member 20 of the medical device 100 may have separate characteristics. The controller 116 also may be coupled either wirelessly or via wire(s) 118 to a display 114 for displaying images received by the medical device 100. The display 114 may include and/or be connected to any suitable input devices, such a touch screen, keyboard, and/or mouse.

The distal end 22 of the elongate member 20 as shown in FIG. 3 also shows that in addition to the optical fibers 150-156, the distal end 22 also may include an imaging device 144, such as a camera configured to capture images for viewing by the user of the medical device 100.

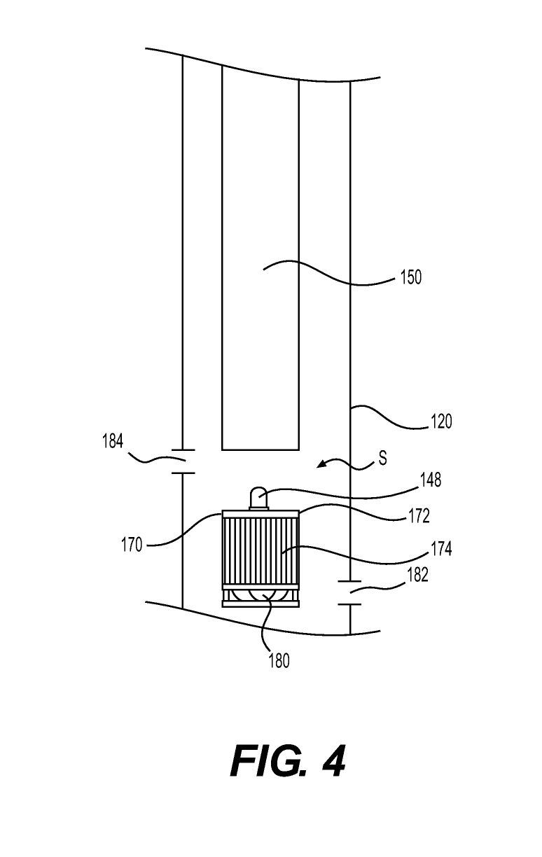

FIG. 4 shows a portion of an elongate member 120 similar to the elongate member 20 of medical device 100. The portion of the elongate member 120 may include an air inlet 182 configured to allow air from outside the medical device 100 to enter a portion of the medical device housing the illumination source 148. The air inlet 182 may be configured to prevent any leakage of fluid from the portion of the elongate member 120, such as via a filter, membrane and/or in any suitable manner. The portion of the elongate member 120 may include the optical fiber 150 made using any suitable materials, such as polymers, silica, or any other suitable materials configured to transmit light. The optical fiber 150 may be disposed in the portion of the elongate member 120 and may have a distal end, and a proximal end positioned a distance S away from the illumination source 148. The distance S may define a space between the illumination source 148 and the proximal end of the optical fiber and ambient air may be forced into this space. For example, the space may be configured to target the conservation of radiance of the system AC) (etendue), while allowing air to cool the system. Thus, the distance S may provide an air space so that the optical fiber 150 and the illumination source 148 may be cooled.

The illumination source 148 may be any suitable source of light, such as an LED of any type (e.g. red, green, white, or blue). The illumination source 148 may be directly coupled to a heat-dissipating member 170 in any suitable manner. For example, the illumination source 148 may include a suitable housing and may be directly disposed on the heat-dissipating member 170 via thermal grease and/or a thermal conductivity pad.

The heat-dissipating member 170 may have any suitable configuration to radiate heat generated by the illumination source 148. For example, the heat-dissipating member 170 may include a surface portion 172 on which the illumination source 148 may be disposed. In some examples, the heat-dissipating member 170 may include a heat sink having one or more fin members 174. The fin members 174 may extend from the surface portion 172 on a side opposite the side of the surface 172 on which the illumination source 148 may be disposed. The fin members 174 may have any suitable size and shape and may be configured to increase the surface area of the heat-dissipating member 170. In some examples, the fin members 174 may be movable and/or automatically adjusted. For example, the speed/direction/duration of the fin members 174 may be based on an automatic electronic processing of the temperature and/or other environmental data (e.g. humidity, moisture) received, for example, from a sensor.

The heat-dissipating member 170 may be manufactured using any suitable materials configured to conduct heat from the illumination source 148, such as metals (e.g. aluminum). In some examples, the heat-dissipating member 170 may be manufactured using cold forged aluminum to optimize thermal flux path to the fin members 174. The heat-dissipating member 170 may be configured to conduct the heat generated from the illumination source 148 and radiate the heat to the rear of the heat-dissipating member 170 via the fin members 174 in any other suitable manner configured to increase the surface area of the heat-dissipating member 170 and/or drawn in and force ambient air through the space between the illumination source 148 and a proximal end of the optical fiber 150. The heat-dissipating member 170 may have any suitable size and shape to dissipate heat generated from the illumination source 148.

The heat radiated by the heat-dissipating member 170 and generated by the illumination source 148 may be moved by a fan 180 or other suitable device configured to generate airflow in a region. The fan 180 may have any suitable size and shape and may include one or more oscillating members such as vanes or blades configured to direct airflow. The oscillating members may rotate or move in any direction in any manner to generate airflow. The fan 180 may include a housing to house the oscillating members. The fan 180 may be positioned at a proximal end of the heat-dissipating member 170. In some examples, the fan 180 may be coupled to a portion of the proximal end of the heat-dissipating member 170. The fan 180 may move heated air from the proximal end of the heat-dissipating member 170, such as via fins 174, and out of the medical device, such as via outlet opening 184. The airflow generated by the fan 180 may reduce the temperature surrounding the heat-dissipating member 170 and the illumination source 148 by generating a greater temperature gradient so that more heat may radiate from the heat-dissipating member 170.

The speed of the oscillating members of the fan 180 may be electronically controlled and optimized via the controller 116 to reduce the temperature in the area of the medical device 100 in which the illumination member 148 is disposed while reducing noise generated by the fan 180. Airflow may be optimized to not only pull air entering through the heat-dissipating member 170, but also to blow over the power supply and LED driver circuits, and out through the outlet 184. Air may be drawn from the ambient area and any increased change in temperature may achieve a cooling effect. Additional fans may be added to direct the air flow in an advantageous manner between the air inlet 182 and outlet 184. The fans may have any suitable size and shape. The larger the temperature gradient between the air and the heat-dissipating member 170, the more efficient the cooling may be. Therefore, it may be advantageous to avoid any trapped air near surfaces of the heat-dissipating member 170. The airflow near the heat-dissipating member 170 may be more important than farther away from the heat-dissipating member 170, as increasing airflow across surfaces of the heat-dissipating member 170 may increase any cooling effect. The speed of the fan 180 also may determine the cooling effect. Although FIG. 4 shows a single optical fiber 150 transmitting light generated by a single illumination source 148 disposed on a single heat-dissipating member 170 and fan 180, the portion of the elongate member 120 may include multiple optical fibers, such as optical fibers 150-156 (as shown in FIG. 3). In some examples, each optical fiber 150-156 may transmit light from the same illumination source. In some examples, each optical fiber 150-156 may transmit light from shared or individual illumination sources 148. In some examples, the illumination sources 148 all may be disposed on a single heat-dissipating member 170 coupled to a single fan 180. Alternatively, each illumination source 148 may be disposed on individual heat-dissipating members 170, each of which may be coupled to a corresponding fan 180. Some optical fibers 150-156 may share a single illumination source, heat-dissipating member 170 and/or fan 180, e.g. optical fibers 150 and 152 may transmit light from first illumination source 148 coupled to a first heat-dissipating member 170 on a first fan 180, and optical fibers 154 and 156 may transmit light from another illumination source coupled to another heat-dissipating member on another fan.

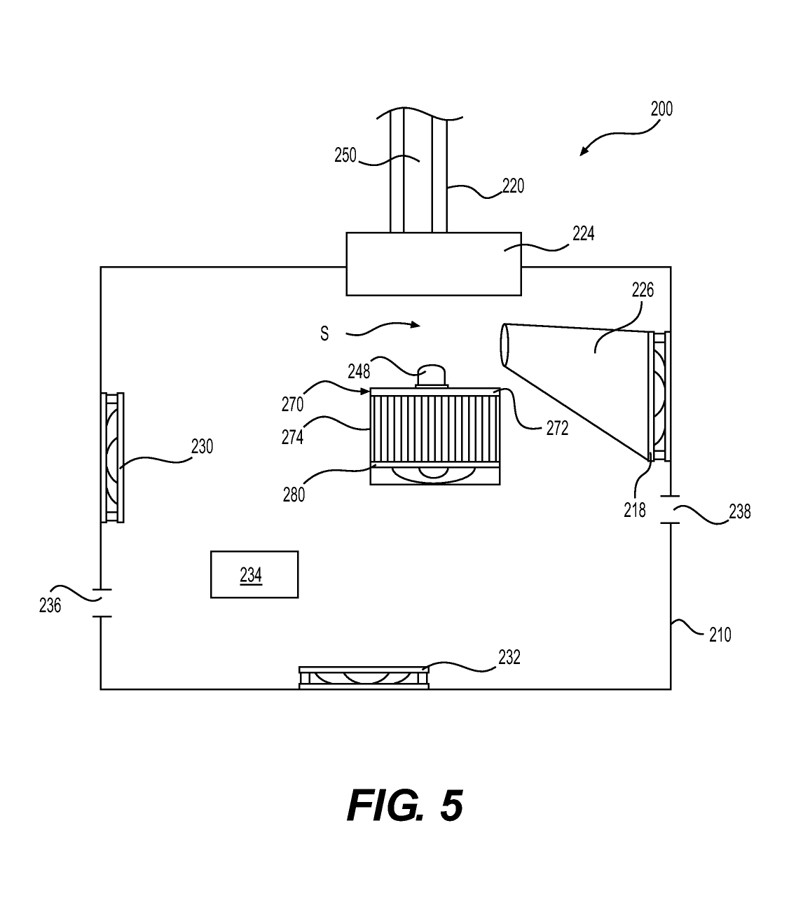

FIG. 5 shows another aspect of dissipating heat generated by an illumination source. FIG. 5 shows a portion 200 of a medical device similar to the medical device 100 in FIG. 1, according to another embodiment. The portion 200 of the medical device, such as device 100 (shown in FIG. 2) may be located at any suitable location of the medical device 100, for example, the portion 200 may be located at a distal end of the medical device 100, such as at distal end 120. Alternatively, the portion may be located in a proximal portion of the medical device 100, such as in a handle portion (not shown) of the medical device. The portion 200 of the medical device 100 may include an elongate member 220, similar to elongate member 20 and may include an optical fiber 250 connected via a connector 224 to a control module 210. The elongate member 220 may be removably or non-removably connected to the connector 224 in any suitable manner, either removably such as via threads or slots. The connector 224 may be removably or non-removably coupled to the control module 210 in any suitable manner. The control module 210 may include one or more illumination sources 248 disposed on a heat-dissipating member 270 in a manner similar to the manner described above in reference to FIG. 4. The illumination source(s) 248 may be separated from connector 224 by a distance S defining a space or a gap. The distance S may be optimized to allow airflow, prevent the optical fiber 250 from making contact with the illumination source 248, and also may conserve any radiance.

The heat-dissipating member 270 may include a surface portion 272 and fin members 274 similar to the heat-dissipating member 170 described in reference to FIG. 4. In addition, a fan 280 may be positioned at a proximal end of the heat-dissipating member 270.

A second fan 218 may be configured to provide forced air into the space S separating the illumination sources 248 and the connector 224. The second fan 218 may include one or more ducts 226 configured to direct the air generated by the second fan 218 into the space and lower the temperature in space. The ducts 226 also may be configured to minimize static pressure and maximize performance and longevity of the fan 218. In addition, the control module 210 may include additional fans 230, 232, which may generate air to circulate in the control module from inlet 238 and toward outlet 236. The control module 210 may include any suitable number of inlets and outlets. The control module 210 also may include a controller 234, which may include a processor and/or sensors, such as temperature and air pressure sensors. The data received from the sensors may be processed by controllers, such as controllers 110 and/or 116 (shown in FIG. 2) and used to optimize use of the illumination source 248 and the fans 280, 218, 230, and 232. The inlet 238 and outlet 236 may be located in close proximity to the fans mounted on the side of the controller 234 (e.g. 230 and 232) such that those fans may move air around the controller 234. The control module 210 also may include one or more optical sensors configured to determine any direct coupling of the connector 224, thus, the illumination source 248 may not be turned on when the connector 224 is not connected (e.g. the illumination source 248 may only be turned on when the connector 224 is connected).

One of the controllers discussed above (e.g. 110, 116, 234) may include or may be connected to a general purpose computer hardware platform and also may be connected to a network or host computer platform (not shown) as may typically be used to implement a server, such as controllers 110 and/or 116, for executing illumination and/or visualization as described above. It is believed that those skilled in the art are familiar with the structure, programming, and general operation of such computer equipment.

The platform may include a data communication interface for packet data communication. The platform may also include a central processing unit (CPU) in the form of one or more processors, for executing program instructions. For example, such platforms may be included in the controllers 110 and/or 116 shown in FIG. 2. The platform typically includes an internal communication bus program storage and data storage for various data files to be processed and/or communicated by the platform such as ROM and RAM, although the server often receives programming and data via network communications. For example, the controllers 110 and/or 116 may receive and/or store data regarding the temperature of the illumination source 248 and surrounding areas (e.g. the space shown in FIG. 5) and may process the temperature data to determine various characteristics of the fans 280, 218, 230, and/or 232.

The hardware elements, operating systems, and programming languages of such equipment are conventional in nature, and it is presumed that those skilled in the art are adequately familiar therewith. The server also may include input and output ports to connect with input and output devices such as keyboards, mice, touchscreens, monitors, displays, etc., such as display 114 shown in FIG. 2. The examples shown in the above figures and described above may force ambient air into the space S between the illumination sources and the heat-dissipating members and also may cool the power architecture of the device and illumination source architecture of the device, and direct air out of the device. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. For example, the controllers, 110, and/or 116 may be in communication with the Internet to send and receive updates to software for controlling the characteristics of the fans 280, 218, 230, and 232.

As used herein, unless restricted to non-transitory, tangible “storage” media, terms such as computer or machine “readable medium” refer to any medium that participates in providing instructions to a processor for execution.

The disclosed medical devices and methods may be utilized in any suitable application involving illumination and/or visualization in the body during a therapeutic and/or diagnostic medical procedure. Any aspect set forth herein may be used with any other aspect set forth herein. The devices may be used in any suitable medical procedure, may be advanced through any suitable body lumen and body cavity. For example, the devices described herein may be used through any natural body lumen or tract, including those accessed orally, vaginally, rectally, nasally, urethrally, or through incisions in any suitable tissue.

It will be apparent to those skilled in the art that various modifications and variations can be made in the disclosed medical devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device configured for insertion into a body, comprising:
an elongate member extending from a proximal end to a distal end, the distal end configured to be positioned inside the body;
an optical fiber disposed in a lumen of the elongate member;
an illumination source configured to emit light through the optical fiber, the illumination source being disposed on a first surface of a heat dissipating member; and
an oscillating member facing a second surface of the heat dissipating member and configured to direct air at the second surface of the heat dissipating member.

2. The medical device of claim 1, further comprising at least one inlet configured to receive air into the medical device and at least one outlet configured to exhaust air from the medical device.

3. The medical device of claim 1, wherein the illumination source comprises at least one LED.

4. The medical device of claim 1 wherein, the heat dissipating member includes one or more protruding members extending between the first surface and the second surface and configured to increase a surface area of the heat dissipating member.

5. The medical device of claim 1, further comprising a control member configured to control characteristics of the oscillating member.

6. The medical device of claim 5, wherein the control member is configured to receive data from a temperature sensor.

7. The medical device of claim 1, wherein a surface of the illumination source is separated from a surface of the optical fiber by a space.

8. The medical device of claim 7, further comprising a second oscillating member configured to direct air in the space separating the surface of the illumination source and the surface of the optical fiber.

9. The medical device of claim 8, wherein the second oscillating member comprises a duct member configured to focus the air in the space separating the surface of the illumination source and the surface of the optical fiber.

10. The medical device of claim 1, further comprising a connector disposed between the optical fiber and the illumination source and coupled to a proximal end of the optical fiber.

11. The medical device of claim 1, wherein the medical device is an endoscope.

12. A medical device configured for insertion into a body, comprising:
an elongate member extending from a proximal end to a distal end, the distal end configured to be positioned inside the body;
an optical fiber disposed in a lumen of the elongate member;
an illumination source configured to emit light through the optical fiber, the illumination source being disposed on a first surface of a heat dissipating member; and
a movable member configured to generate and direct air in a space between the illumination source and the optical fiber.

13. The medical device of claim 12, wherein the heat dissipating member comprises one or more protruding members extending between the first surface and a second surface and configured to increase a surface area of the heat dissipating member.

14. The medical device of claim 12, further comprising a duct coupled to the movable member and configured to focus the air generated by the movable member.

15. The medical device of claim 12, further comprising at least one inlet configured to receive air into the medical device and at least one outlet configured to exhaust air from the medical device.

16. The medical device of claim 12, further comprising a control member configured to control characteristics of the oscillating member.

17. The medical device of claim 16, wherein the control member is configured to receive data from a temperature sensor.

18. The medical device of claim 17, further comprising a second oscillating member disposed at an end portion of the heat-dissipating member and configured to direct air at the heat-dissipating member.

19. The medical device of claim 17, further comprising a connector disposed between the optical fiber and the illumination source and coupled to a proximal end of the optical fiber.

20. A method of dissipating heat from an illuminating medical device comprising:
receiving air from outside the medical device through an inlet;
generating a first airflow path from a first oscillating device to a first surface of an illumination source;
generating a second airflow path from a second oscillating device to a space between a second surface of the illumination source and an optical fiber; and
expelling air from the medical device.
